(19) United States
(12) Patent Application Publication
(10) Pub. No.: US 2005/0149017 A1
(43) Pub. Date: Jul. 7, 2005

(54) Movable handle for vessel sealer

(76) Inventor: Sean T. Dycus, Denver, CO (US)

Correspondence Address: Mark Farber, Esq.;United States Surgical,, A Division of Tyco Healthcare Group, LP, 150 Glover Avenue, Norwalk, CT, 06856, US

(21) Appl. No.: 10/991,975

(22) Filed: Nov. 18, 2004

Related U.S. Application Data
(60) Provisional application No. 60/523,128, filed on Nov. 18, 2003.

(51) Int. Cl. A61B 18/14

(52) U.S. Cl. 606/51

(57) ABSTRACT

Bipolar forceps are provided including a shaft having opposing jaw members at a distal end thereof, the shaft defining a longitudinal axis; a drive assembly for moving the jaw members relative to one another from a first position wherein the jaw members are disposed in spaced relation relative to one another to a second position wherein the jaw members cooperate to grasp tissue therebetween; and a handle assembly operatively connected to a proximal end of the shaft. The handle assembly includes a first handle and a second handle, wherein the handle assembly is pivotable between a first position in which handle assembly is angled with respect to the longitudinal axis and a second position in which the handle assembly is substantially axially aligned with the longitudinal axis.

### CROSS-REFERENCE TO RELATED APPLICATIONS

The present application claims the benefit of and priority to U.S. Provisional Application Ser. No. 60/523,128, filed on Nov. 18, 2003, the entire disclosure of which is incorporated herein by reference.

### BACKGROUND

1. Technical Field

The present disclosure relates to electrosurgical instruments and methods for performing endoscopic surgical procedures and, more particularly, to open and/or endoscopic bipolar electrosurgical forceps and methods for sealing and/or cutting tissue.

2. Background of Related Art

A hemostat or forceps is a simple pliers-like tool which uses mechanical action between its jaws to constrict vessels and is commonly used in open surgical procedures to grasp, dissect and/or clamp tissue. Electrosurgical forceps utilize both mechanical clamping action and electrical energy to effect hemostasis by heating the tissue and blood vessels to coagulate, cauterize and/or seal tissue.

Over the past several decades, an increased number of surgeons are complementing traditional open methods of gaining access to vital organs and body cavities with endoscopes and endoscopic instruments which access organs through small puncture-like incisions. Endoscopic instruments are inserted into the patient through a cannula, or port, which has been made with a trocar. Certain endoscopic surgical procedures require cutting blood vessels or vascular tissue. However, due to space limitations, surgeons can have difficulty suturing vessels or performing other traditional methods of controlling bleeding, e.g., clamping and/or tying-off transected blood vessels. Blood vessels, in the range below two millimeters, can often be closed using standard electrosurgical techniques. However, if a larger vessel is severed, it may be necessary for the surgeon to convert the endoscopic procedure into an open-surgical procedure and thereby abandon the benefits of laparoscopy.

By utilizing electrosurgical forceps, a surgeon can cauterize, coagulate/desiccate and/or simply reduce or slow bleeding, by controlling the intensity, frequency and duration of the electrosurgical energy applied through the jaw members to the tissue. The electrode of each jaw member is charged to a different electric potential such that when the jaw members grasp tissue, electrical energy can be selectively transferred through the tissue.

In order to effect a proper seal with larger vessels, two predominant mechanical parameters need to be controlled accurately, namely, the pressure applied to the vessel, and the gap distance between the electrodes of the jaw members, both of which are affected by the thickness of the vessel to be sealed. More particularly, accurate application of pressure is important to oppose the walls of the vessel; to reduce the tissue impedance to a low enough value that allows enough electrosurgical energy through the tissue; to overcome the forces of expansion during tissue heating; and to contribute to the end tissue thickness which is an indication of a good seal. It has been determined that a typical fused vessel wall is optimum between 0.001 and 0.006 inches. Below this range, the seal may shred or tear and above this range the lumens may not be effectively sealed.

It is known that it is difficult to adequately control thickness of the resulting sealed tissue by controlling clamping pressure alone for either of two reasons, namely, if too much force is applied, there is a possibility that the two electrodes (i.e., the two poles) will touch and energy will not be transferred through the tissue resulting in an ineffective seal; or if too low a force is applied the tissue may pre-maturely move prior to activation and sealing and/or a thicker, less reliable seal may be created.

As mentioned above, in order to properly and effectively seal larger vessels, a greater force between the opposing jaw members of the electrosurgical forceps is required. As a result thereof, providing an electrosurgical instrument which consistently provides the appropriate closure force between opposing electrodes within a preferred pressure range will enhance the chances of a successful seal. As can be appreciated, the appropriate closure force between the opposing electrodes of the electrosurgical forceps often depends on the manual strength and/or the position/orientation of the surgeons hand in order to provide the appropriate closure force within the appropriate range on a consistent basis. Additionally, the overall success of creating an effective tissue seal is greatly reliant upon the surgeon's expertise, vision, dexterity and experience in judging the appropriate closure force to uniformly, consistently and effectively seal the vessel. In other words, the success of the seal would greatly depend upon the ultimate skill of the surgeon rather than the efficiency of the instrument.

As can further be appreciated, during a given surgical procedure, one difficulty that may often arise for a surgeon is that the electrosurgical instrument may become somewhat unwieldy, cumbersome and/or difficult to actuate in a given position, i.e., the trigger may be positioned in an awkward location and orientation thus making it difficult to grasp. For example, during an endoscopic surgical procedure where the cannula channel has a vertical axis, a surgeon may not be able to easily manipulate a pistol grip-type instrument and actuate the trigger without inadvertently changing the desired position (or angle) of the electrosurgical instrument (e.g., forceps), in particular, the end effectors (e.g., the jaw members).

Accordingly, there exists a need to develop an electrosurgical instrument which is selectively configurable and/or adaptable for operation in a number of different orientations and configurations.

### SUMMARY

The present disclosure relates to a bipolar forceps which includes a shaft having opposing jaw members at a distal end thereof and a drive assembly for moving the jaw members relative to one another from a first position, wherein the jaw members are disposed in spaced relation relative to one another, to a second position, wherein the jaw members cooperate to grasp tissue therebetween. The forceps also includes a multi-position handle assembly which is pivotable between a first and at least a second gripping position for actuating the drive assembly to move the jaw members. The forceps are connected to a source of electrosurgical energy such that the jaw members are capable of conducting energy through tissue held therebetween to effect a tissue seal.

A rotating assembly may also be included for rotating the jaw members about a longitudinal axis defined through the shaft.

According to one aspect of the present disclosure, bipolar forceps are provided. The bipolar forceps includes a shaft having opposing jaw members at a distal end thereof, the shaft defining a longitudinal axis; a drive assembly for moving the jaw members relative to one another from a first position wherein the jaw members are disposed in spaced relation relative to one another to a second position wherein the jaw members cooperate to grasp tissue therebetween; and a handle assembly operatively connected to a proximal end of the shaft. The handle assembly includes a first handle and a second handle, wherein the handle assembly is pivotable between a first position in which handle assembly is angled with respect to the longitudinal axis and a second position in which the handle assembly is substantially axially aligned with the longitudinal axis.

It is envisioned that the second handle is selectively movable from a first position in spaced relation relative to the first handle and a second position in relatively close proximity to the first handle. Desirably, movement of the second handle between the first and second positions imparts movement of the jaw members between the first and second positions.

In one embodiment, the forceps further includes a rotating assembly for rotating the jaw members about the longitudinal axis. The forceps may further include a source of electrical energy connected to each jaw member such that the jaw members are capable of conducting energy through tissue held therebetween to effect a seal.

In another embodiment, the forceps includes a generally tube-like cutter slidably engaged about said elongated shaft which is selectively movable about the elongated shaft to engage and cut tissue on at least one side of the jaw members while the tissue is engaged between the jaw members.

The forceps may include opposing electrically conductive sealing surfaces disposed on inner facing surfaces of the jaw members. Desirably, at least one of the jaw members is made from a hard anodized aluminum having high dielectric properties. It is envisioned that the electrically conductive sealing surfaces include a non-stick coating disposed thereon which is designed to reduce tissue adherence.

The handle assembly is dimensioned to maintain a closure pressure in the range of about 3 kg/cm² to about 16 kg/cm² between the electrically conductive sealing surfaces. The forceps may further include at least one non-conductive stop which creates a gap between the electrically conductive sealing surfaces within the range of about 0.001 inches to about 0.006 inches.

According to another aspect of the present disclosure, an electrosurgical instrument for performing at least one of sealing and dividing tissue, is provided. The electrosurgical instrument includes a housing having a shaft attached thereto, the shaft defining a longitudinal axis; a first jaw member movable relative to a second jaw member, the first jaw member attached to the shaft and being relatively movable from a first open position wherein the jaw members are disposed in spaced relation relative to one another to a second closed position wherein the jaw members cooperate to grasp tissue therebetween; a drive rod assembly for imparting movement of the jaw members between the first and second positions; a rotating assembly attached to the housing for rotating the jaw members about the longitudinal axis; a knife assembly attached to the housing for separating tissue grasped between the jaw members; and a handle assembly pivotally attached to the housing for actuating the drive rod assembly.

The handle assembly includes a first handle and a second handle, wherein the handle assembly is pivotable between a first position in which handle assembly is angled with respect to the longitudinal axis and a second position in which the handle assembly is substantially axially aligned with the longitudinal axis. Desirably, the second handle is selectively movable from a first position in spaced relation relative to the first handle and a second position in relatively close proximity to the first handle. In use, movement of the second handle between the first and second positions imparts movement of the jaw members between the first and second positions.

The electrosurgical instrument further includes first and second electrical leads which connect the jaw members to a source of electrical energy such that the jaw members are capable of conducting energy through tissue held therebetween; and a handswitch attached to the housing which allows a user to selectively energize the jaw members.

The electrosurgical instrument may include a source of electrical energy connected to each jaw member such that the jaw embers are capable of conducting energy through tissue held therebetween to effect a seal.

It is envisioned that the electrosurgical instrument may include a generally tube-like cutter slidably engaged about said elongated shaft which is selectively movable about the elongated shaft to engage and cut tissue on at least one side of the jaw members while the tissue is engaged between the jaw members.

The electrosurgical instrument further includes opposing electrically conductive sealing surfaces disposed on inner facing surfaces of the jaw members. Desirably, at least one of the jaw members is made from a hard anodized aluminum having high dielectric properties.

The handle assembly is desirably dimensioned to maintain a closure pressure in the range of about 3 kg/cm² to about 16 kg/cm² between the electrically conductive sealing surfaces. The electrosurgical instrument further includes at least one non-conductive stop which creates a gap between the electrically conductive sealing surfaces within the range of about 0.001 inches to about 0.006 inches.

### BRIEF DESCRIPTION OF THE DRAWINGS

Various embodiments of the subject instrument are described herein with reference to the drawings wherein:

FIG. 1A is a left, perspective view of a prior art endoscopic bipolar forceps showing a housing, a shaft and an end effector assembly;

FIG. 1B is a left, perspective view of a prior art open bipolar forceps with pistol grip;

FIG. 2 is a side elevational view of an endoscopic forceps, according to the present disclosure, shown in a pistol grip-type configuration;

FIG. 3 is a side elevational view of the endoscopic forceps of FIG. 2 shown in a handshake grip-type configuration;

FIG. 4 is an enlarged, left perspective view of the end effector of the endoscopic forceps of FIGS. 2 and 3; and

FIG. 5 is an enlarged, right side elevational view of the end effector of the endoscopic forceps of FIGS. 2 and 3.

### DETAILED DESCRIPTION OF PREFERRED EMBODIMENTS

Preferred embodiments of the presently disclosed instruments will now be described in detail with reference to the drawing figures wherein like reference numerals identify similar or identical elements. In the drawings and in the description which follows, the term “proximal”, as is traditional, will refer to the end of the instrument which is furthest from the operator and the term “distal” will refer to the end of the instrument which is closest to the operator.

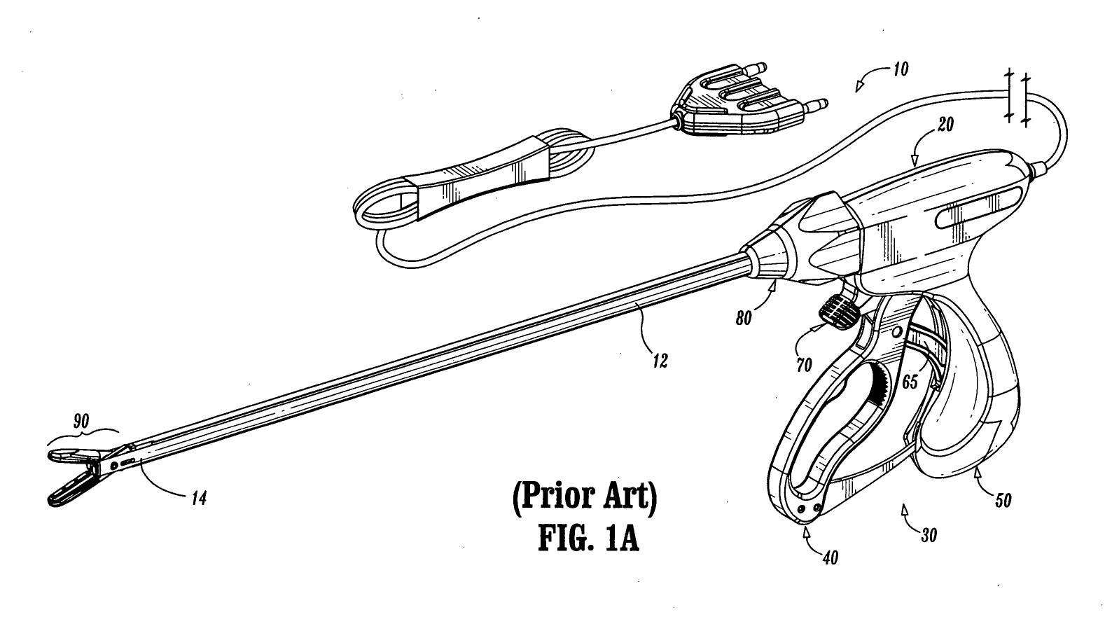

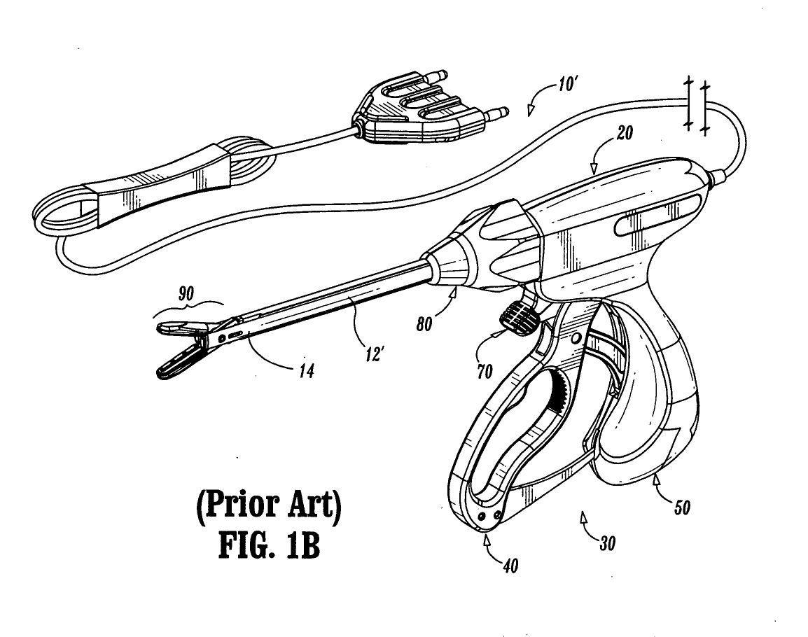

Referring to FIGS. 1A and 1B, prior art bipolar forceps, for use in various endoscopic and/or open surgical procedures, are shown. As seen in FIG. 1A, an endoscopic bipolar forceps is shown generally as 10 and, as seen in FIG. 1B, an open bipolar forceps is shown generally as 10′. Forceps 10 and 10′ include a housing 20, a handle assembly 30, a rotating assembly 80, a trigger assembly 70 and an end effector assembly 90 which mutually cooperate to grasp, seal and divide tubular vessels and vascular tissue.

As seen in FIGS. 1A and 1B, handle assemblies 30 of forceps 10 and 10′ are of the pistol grip-type. Accordingly, such a handle assembly 30 offers the surgeon a single gripping position from which to grasp forceps 10, 10′ and to transmit a clamping pressure to end effector 90.

In addition, each forceps 10 and 10′ includes a shaft 12 and 12′, respectively, which have a distal end 14 configured and dimensioned to mechanically engage end effector assembly 90 and a proximal end 16 configured and dimensioned to mechanically engage housing 20. As seen in FIGS. 1A and 1B, shaft 12 of forceps 10 is relatively longer than shaft 12′ of forceps 10′. The relatively longer shaft 12 of forceps 10 enables forceps 10 to be used in performing endoscopic surgical procedures.

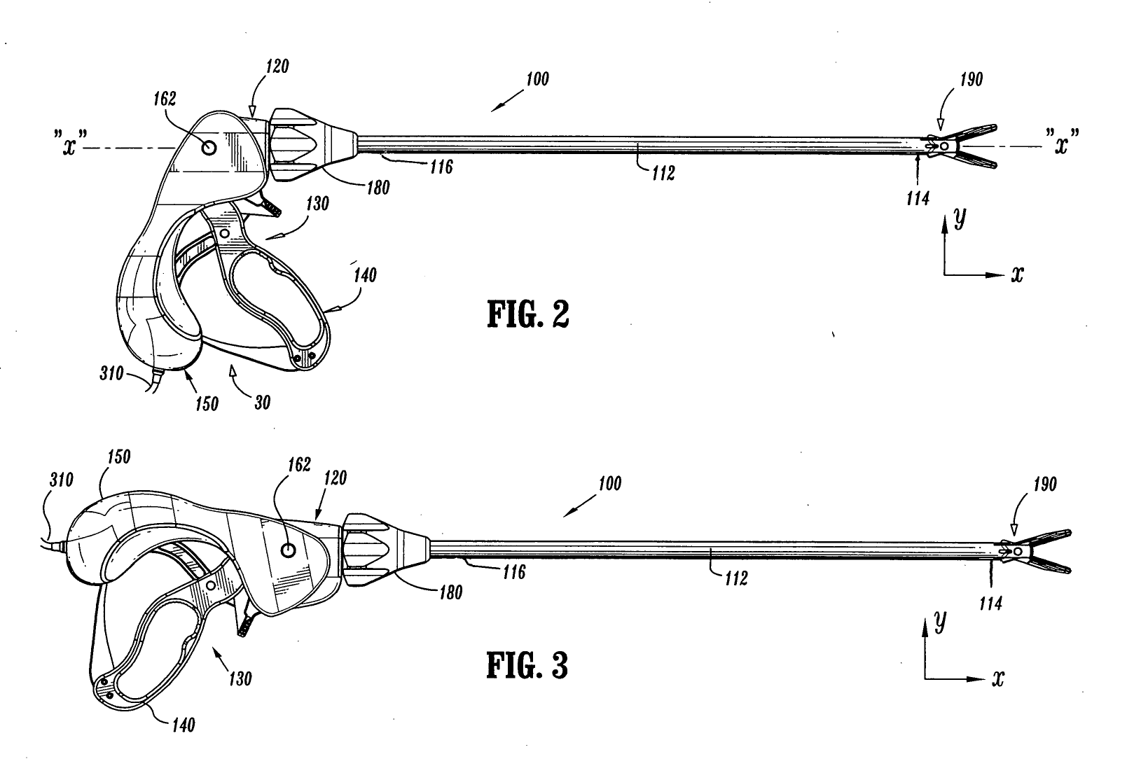

Turning now to FIGS. 2 and 3, a bipolar forceps in accordance with an embodiment of the present disclosure is shown generally as forceps 100. Although the majority of the remaining figure drawings depict an endoscopic bipolar forceps 100 for use in connection with endoscopic surgical procedures, an open bipolar forceps is also contemplated for use in connection with traditional open surgical procedures. For the purposes herein, the endoscopic version of forceps 100 is discussed in detail, however, it is contemplated that the open forceps also include the same or similar operating components and features as described below.

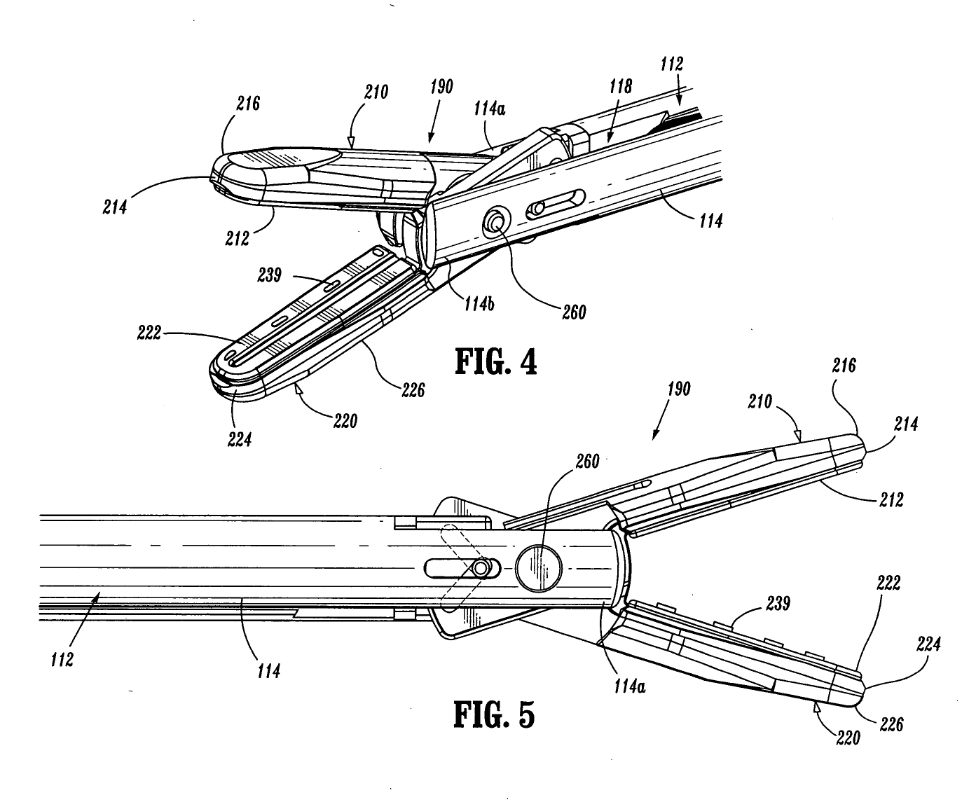

Forceps 100 includes a housing 120, a multi-position handle assembly 130, a rotating assembly 180, a trigger assembly 170 and an end effector assembly 190 which mutually cooperate to grasp, seal and divide tubular vessels and vascular tissue. Forceps 100 further includes a shaft 112 which has a distal end 114 dimensioned to mechanically engage end effector assembly 190 and a proximal end 116 which mechanically engages housing 120. Preferably, as seen in FIGS. 4 and 5, shaft 112 is bifurcated at distal end 114 thereof to form ends 114a and 114b which are dimensioned to receive end effector assembly 190.

Forceps 100 preferably includes a rotating assembly 180. Rotating assembly 180 is attached to a distal end of housing 120 and is rotatable approximately 180 degrees in either direction about a longitudinal axis “X”. Rotation of rotating assembly 180 correspondingly rotates end effector assembly 190 about axis “X”.

As seen in FIGS. 2 and 3, forceps 100 further includes an electrical cable 310 extending from handle assembly 130 which connects forceps 100 to a source of electrosurgical energy, e.g., a generator (not shown). Preferably, generators such as those sold by Valleylab—a division of Tyco Healthcare LP, located in Boulder, Colo., are used as a source of electrosurgical energy, e.g., FORCE EZ™ Electrosurgical Generator, FORCE FX™ Electrosurgical Generator, FORCE 1C™, FORCE 2™ Generator, SurgiStat™ II. One such system is described in commonly-owned U.S. Pat. No. 6,033,399 entitled “ELECTROSURGICAL GENERATOR WITH ADAPTIVE POWER CONTROL” the entire contents of which are hereby incorporated by reference herein. Other systems have been described in commonly-owned U.S. Pat. No. 6,187,003 entitled “BIPOLAR ELECTROSURGICAL INSTRUMENT FOR SEALING VESSELS” the entire contents of which is also incorporated by reference herein.

Preferably, the generator includes various safety and performance features including isolated output, independent activation of accessories, and the Valleylab REM™ Contact Quality Monitoring System, which may substantially reduce the risk of burns under the patient return electrode. Preferably, the electrosurgical generator includes Valleylab's Instant Response™ technology features which provide an advanced feedback system which senses changes in tissue 200 times per second and adjusts voltage and current to maintain appropriate power. The Instant Response™ technology is believed to provide one or more of the following benefits to surgical procedures:
Consistent clinical effect through all tissue types.
Reduced thermal spread and risk of collateral tissue damage.
Less need to “turn up the generator”.
Designed for the minimally invasive environment.

Handle assembly 130 includes a first handle 150 and a second handle 140. As best seen in FIGS. 2 and 3, handle assembly 130 is pivotable about a pivot 162 from a first position in which forceps 100 is in a pistol grip-type configuration (i.e., handle assembly 130 is angled with respect to the longitudinal “X” axis) and a second position in which forceps 100 is in a handshake grip-type configuration or longitudinally-aligned configuration (i.e., handle assembly 130 is aligned with respect to the longitudinal “X” axis). Preferably, the axis of rotation of pivot 162 is substantially orthogonal to the longitudinal “X” axis. In addition, second handle 140 is selectively movable about a pivot (not shown) from a first position in spaced relation relative to first handle 150 to a second position in closer proximity relative to first handle 150 which, as explained in greater detail below, imparts movement of jaw members 210, 220 relative to one another from an open to closed position about tissue.

Turning to FIGS. 4 and 5, end effector assembly 190 is attached to distal end 114 of shaft 112 and includes a pair of opposing jaw members 210 and 220. First and second handles 140, 150 are ultimately connected to a drive rod (not shown) which, together, mechanically cooperate to impart movement of jaw members 210, 220 from an open position wherein the jaw members 210, 220 are disposed in spaced relation relative to one another, to a clamping or closed position wherein jaw members 210, 220 cooperate to grasp tissue therebetween.

As best seen in FIGS. 4 and 5, jaw members 210 and 220 are seated within a cavity 118 defined between bifurcated ends 114a and 114b of shaft 112. Jaw members 210 and 220 are generally symmetrical and include similar component features which cooperate to permit facile rotation about a pivot pin 260 to effect the sealing and dividing of tissue. As a result and unless otherwise noted, only jaw member 210 and the operative features associated therewith are described in detail herein, but as can be appreciated, many of these features apply to jaw member 220 as well.

Jaw member 210 includes a jaw housing 216, an insulative substrate or insulator 214 and an electrically conductive surface 212. Insulator 214 is preferably dimensioned to securely engage the electrically conductive sealing surface 212. This may be accomplished by stamping, by overmolding, by overmolding a stamped electrically conductive sealing plate and/or by overmolding a metal injection molded seal plate.

All of these manufacturing techniques produce an electrode having an electrically conductive sealing surface 212 which is substantially surrounded by an insulating substrate 214. Insulating substrate 214, electrically conductive sealing surface 212 and the outer, non-conductive jaw housing 216 are preferably dimensioned to limit and/or reduce many of the known undesirable effects related to tissue sealing, e.g., flashover, thermal spread and stray current dissipation. Alternatively, it is also envisioned that jaw member 210 and 220 may be manufactured from a ceramic-like material and electrically conductive surface(s) 212 are coated onto the ceramic-like jaw members 210, 220.

It is envisioned that electrically conductive sealing surface 212 may also include an outer peripheral edge which has a radius and insulator 214 meets electrically conductive sealing surface 212 along an adjoining edge which is generally tangential to the radius and/or meets along the radius. Preferably, at the interface, electrically conductive sealing surface 212 is raised relative to insulator 214. These and other envisioned embodiments are discussed in commonly assigned International Application Serial No. PCT/US01/11412 entitled “ELECTROSURGICAL INSTRUMENT WHICH REDUCES COLLATERAL DAMAGE TO ADJACENT TISSUE” by Johnson et al., and commonly assigned International Application Serial No. PCT/US01/11411 entitled “ELECTROSURGICAL INSTRUMENT WHICH IS DESIGNED TO REDUCE THE INCIDENCE OF FLASHOVER” by Johnson et al.

Preferably, jaw members 210, 220 are electrically isolated from one another such that electrosurgical energy can be effectively transferred through the tissue to form the seal.

Preferably, electrically conductive sealing surfaces 212, 222 of jaw members 210, 220, respectively, are relatively flat to avoid current concentrations at sharp edges and to avoid arcing between high points. In addition, and due to the reaction force of the tissue when engaged, jaw members 210, 220 are preferably manufactured to resist bending. For example, jaw members 210, 220 may be tapered along the width thereof which is advantageous for two reasons: 1) the taper will apply constant pressure for a constant tissue thickness at parallel; and 2) the thicker proximal portion of jaw members 210, 220 will resist bending due to the reaction of the tissue.

It is also envisioned that jaw members 210, 220 may be curved in order to reach specific anatomical structures. For example, it is contemplated that dimensioning jaws 210, 220 at an angle of about 50 degrees to about 70 degrees is preferred for accessing and sealing specific anatomical structures relevant to prostatectomies and cystectomies, e.g., the dorsal vein complex and the lateral pedicles.

As best seen in FIGS. 4 and 5, in order to achieve a desired gap range (e.g., about 0.001 to about 0.006 inches) and apply a desired force to seal the tissue, at least one jaw member 210 and/or 220 includes at least one stop member 239 which limits the movement of the two opposing jaws 210, 220 relative to one another. Preferably, each stop member 239 is made from an insulative material and is dimensioned to limit opposing movement of jaw members 210, 220 to within the above gap range.

Housing 120 is preferably formed from two housing halves which engage one another via a series of mechanical interfaces to form an internal cavity for housing the internal working components of forceps 100. For the purposes herein, the housing halves are generally symmetrical and, unless otherwise noted, a component described with respect to a first of the housing halves will have a similar component which forms a part of a second of the housing halves.

As mentioned above, first handle 150 and second handle 140 of handle assembly 130 cooperate with one another and with housing 120 to activate a first mechanical linkage (not shown) which, in turn, actuates a drive assembly (not shown) for imparting movement of opposing jaw members 210, 220 relative to one another to grasp tissue therebetween.

Handle assembly 130 further includes a second mechanical linkage (not shown) which enables pivoting of first and second handles 150, 140 from the first configuration to the second configuration and which enables first and second handles 150, 140 to impart movement of jaw members 210, 220, relative to one another to grasp tissue therebetween, while in at least one of the first or second configurations.

Forceps 100 can further be provided with a locking mechanism (not shown) which is configured and adapted to selectively fix the position of handle assembly 130 in at least the first and second configurations. In this manner, when the surgeon is prepared to squeeze handle assembly 130 (i.e., approximate first handle 150 and second handle 140 toward one another) to approximate jaw members 210, 220, handle assembly 130 is in a fixed position relative to housing 120. It is further envisioned that the locking mechanism can act as a ratchet like mechanism and enable handle assembly 130 to be selectively fixed to any one of a number of angles relative to the longitudinal “X” axis.

As can be appreciated, handling of forceps 100 while in the pistol grip-type configuration facilitates repositioning of the trajectory of forceps 100 within the surgical area, namely, facilitates axial rotation about the longitudinal “X” axis. Meanwhile, handling of forceps 100 while in the handshake grip-type configuration facilitates repositioning of the trajectory of forceps 100 within the surgical area, namely, facilitates orbital motion in the X-Y plane and the X-Z plane, and about the X axis.

As discussed above, by controlling the intensity, frequency and duration of the electrosurgical energy applied to the tissue, the surgeon can either cauterize, coagulate/desiccate, seal and/or simply reduce or slow bleeding. In addition, two mechanical factors play an important role in determining the resulting thickness of the sealed tissue and effectiveness of the seal, namely, the pressure applied between opposing jaw members 210, 220, and the gap distance between opposing sealing surfaces 212, 222 of jaw members 210, 220, respectively.

However, the thickness of the resulting tissue seal cannot be adequately controlled by force alone. For example, too much force and jaw members 210, 220 would touch and possibly short resulting in little energy traveling through the tissue thus resulting in no tissue sealing, too little force and jaw members 210, 220 would be too far apart and could result in a relatively thick tissue seal.

Applying the correct force is also important for other reasons, namely, to oppose the walls of the vessel, to reduce the tissue impedance to a low enough value that allows enough electrosurgical current through the tissue, and to overcome the forces of expansion during tissue heating in addition to contributing towards creating the required end tissue thickness which is an indication of a good seal.

Experimental results suggest that the magnitude of pressure exerted on the tissue by tissue contacting sealing surfaces 212, 222 is important in assuring a proper surgical outcome. Tissue pressures within a working range of about 3 kg/cm² to about 16 kg/cm² and, preferably, within a working range of 7 kg/cm² to about 13 kg/cm² have been shown to be effective for sealing arteries and vascular bundles.

It is envisioned that forceps 100 may be designed such that it is fully or partially disposable depending upon a particular purpose or to achieve a particular result. For example, end effector assembly 190 may be selectively and releasably engagable with distal end 114 of shaft 112 and/or proximal end 116 of shaft 112 may be selectively and releasably engagable with housing 120 and handle assembly 130. In either of these two instances, forceps 100 would be considered “partially disposable” or “reposable”, i.e., a new or different end effector assembly 190 (or end effector assembly 190 and shaft 112) selectively replaces the old and/or used end effector assembly 190 as needed.

From the foregoing and with reference to the various figure drawings, those skilled in the art will appreciate that certain modifications can also be made to the present disclosure without departing from the scope of the present disclosure.

For example, as disclosed herein, forceps 100 operate by transferring electrosurgical energy through opposing electrically conductive sealing surfaces having different electrical potentials to effect vessel sealing. However, it is also contemplated that the instruments discussed and described herein may be designed and constructed to seal the tissue structure using so-called “resistive heating” and/or radio frequency (RF) energy.

It has been determined that by controlling the RF energy and the pressure, and by maintaining a gap distance in the range of about 0.001 inches to about 0.006 inches between tissue contacting sealing surfaces 212 and 222, effective and consistent tissue sealing may be achieved in a broad range of tissue types.

It is further envisioned that forceps 100 may employ any combination of one or more of the above heating technologies and a switch (not shown) which allows the surgeon the option of the different heating technologies.

Materials used to construct the individual components of forceps 100 may be chosen from a variety of known materials to achieve the desired results. In addition, first handle 140 and second handle 150 may be designed to include a variety of ergonomically pleasing features to enhance the overall “feel” of forceps 100 during handling and use. For example, handles 140 and/or 150 may include a variety of scallops and/or curves which substantially contour the hand of the surgeon. In addition, handles 140, 150 may include rubber-like surfaces to enhance the surgeon's grip during use.

While several embodiments of the disclosure have been shown in the drawings, it is not intended that the disclosure be limited thereto, as it is intended that the disclosure be as broad in scope as the art will allow and that the specification be read likewise.

A more detailed description of the various elements and components of bipolar forceps 100 may be found in U.S. patent application Ser. No. 2003/0188294, filed Apr. 5, 2002, entitled “Vessel Sealer and Divider”; U.S. patent application Ser. No. 2003/0018331, filed Jun. 25, 2002, entitled “Vessel Sealer and Divider”; U.S. patent application Ser. No. 2003/0229344, filed Feb. 20, 2003, entitled “Vessel Sealer and Divider and Method of Manufacturing Same”; U.S. patent application Ser. No. 2004/0082952, filed Apr. 6, 2001, entitled “Vessel Sealer and Divider”; U.S. patent application Ser. No. 2004/0087943, filed Apr. 6, 2001, entitled “Vessel Sealer and Divider”; U.S. patent application Ser. No. 2004/0116924, filed Apr. 6, 2001, entitled “Vessel Sealer and Divider”; International Application No. PCT/US01/11224, filed Apr. 6, 2001, entitled “Vessel Sealer and Divider”; International Application No. PCT/US01/11225, filed Apr. 6, 2001, entitled “Vessel Sealer and Divider”; and International Application No. PCT/US02/01890, filed Jan. 22, 2002, entitled “Vessel Sealer and Divider”; the entire contents of each of which being incorporated herein by reference.

Therefore, the above description should not be construed as limiting, but merely as exemplifications of preferred embodiments. Those skilled in the art will envision other modifications within the scope and spirit of the claims appended hereto.

1. A bipolar forceps, comprising:
a shaft having opposing jaw members at a distal end thereof, the shaft defining a longitudinal axis;
a drive assembly for moving the jaw members relative to one another from a first position wherein the jaw members are disposed in spaced relation relative to one another to a second position wherein the jaw members cooperate to grasp tissue therebetween; and
a handle assembly operatively connected to a proximal end of the shaft, the handle assembly including:
a first handle and a second handle, wherein the handle assembly is pivotable between a first position in which handle assembly is angled with respect to the longitudinal axis and a second position in which the handle assembly is substantially axially aligned with the longitudinal axis.

2. The forceps according to claim 1, wherein the second handle is selectively movable from a first position in spaced relation relative to the first handle and a second position in relatively close proximity to the first handle.

3. The forceps according to claim 2, wherein movement of the second handle between the first and second positions imparts movement of the jaw members between the first and second positions.

4. The forceps according to claim 3, further comprising a rotating assembly for rotating the jaw members about the longitudinal axis.

5. The forceps according to claim 4, further comprising a source of electrical energy connected to each jaw member such that the jaw members are capable of conducting energy through tissue held therebetween to effect a seal.

6. The forceps according to claim 5, further comprising a generally tube-like cutter slidably engaged about said elongated shaft which is selectively movable about the elongated shaft to engage and cut tissue on at least one side of the jaw members while the tissue is engaged between the jaw members.

7. The forceps according to claim 6, further comprising opposing electrically conductive sealing surfaces disposed on inner facing surfaces of the jaw members.

8. The forceps according to claim 7, wherein at least one of the jaw members is made from a hard anodized aluminum having high dielectric properties.

9. The forceps according to claim 8, wherein the handle assembly is dimensioned to maintain a closure pressure in the range of about 3 kg/cm² to about 16 kg/cm² between the electrically conductive sealing surfaces.

10. The forceps according to claim 9, wherein the electrically conductive sealing surfaces include a non-stick coating disposed thereon which is designed to reduce tissue adherence.

11. The forceps according to claim 10, further comprising at least one non-conductive stop which creates a gap between the electrically conductive sealing surfaces within the range of about 0.001 inches to about 0.006 inches.

12. An electrosurgical instrument for performing at least one of sealing and dividing tissue, comprising:
a housing having a shaft attached thereto, the shaft defining a longitudinal axis;
a first jaw member movable relative to a second jaw member, the first jaw member attached to the shaft and being relatively movable from a first open position wherein the jaw members are disposed in spaced relation relative to one another to a second closed position wherein the jaw members cooperate to grasp tissue therebetween;
a drive rod assembly for imparting movement of the jaw members between the first and second positions;
a rotating assembly attached to the housing for rotating the jaw members about the longitudinal axis;
a knife assembly attached to the housing for separating tissue grasped between the jaw members;
a handle assembly pivotally attached to the housing for actuating the drive rod assembly, the handle assembly including:
a first handle and a second handle, wherein the handle assembly is pivotable between a first position in which handle assembly is angled with respect to the longitudinal axis and a second position in which the handle assembly is substantially axially aligned with the longitudinal axis;
first and second electrical leads which connect the jaw members to a source of electrical energy such that the jaw members are capable of conducting energy through tissue held therebetween; and
a handswitch attached to the housing which allows a user to selectively energize the jaw members.

13. The electrosurgical instrument according to claim 12, further comprising a source of electrical energy connected to each jaw member such that the jaw embers are capable of conducting energy through tissue held therebetween to effect a seal.

14. The electrosurgical instrument according to claim 13, wherein the second handle is selectively movable from a first position in spaced relation relative to the first handle and a second position in relatively close proximity to the first handle.

15. The electrosurgical instrument according to claim 14, wherein movement of the second handle between the first and second positions imparts movement of the jaw members between the first and second positions.

16. The electrosurgical instrument according to claim 15, further comprising a generally tube-like cutter slidably engaged about said elongated shaft which is selectively movable about the elongated shaft to engage and cut tissue on at least one side of the jaw members while the tissue is engaged between the jaw members.

17. The electrosurgical instrument according to claim 16, further comprising opposing electrically conductive sealing surfaces disposed on inner facing surfaces of the jaw members.

18. The electrosurgical instrument according to claim 17, wherein at least one of the jaw members is made from a hard anodized aluminum having high dielectric properties.

19. The electrosurgical instrument according to claim 18, wherein the handle assembly is dimensioned to maintain a closure pressure in the range of about 3 kg/cm² to about 16 kg/cm² between the electrically conductive sealing surfaces.

20. The electrosurgical instrument according to claim 19, further comprising at least one non-conductive stop which creates a gap between the electrically conductive sealing surfaces within the range of about 0.001 inches to about 0.006 inches.

21. A bipolar forceps, comprising:
a shaft having opposing jaw members at a distal end thereof and a drive assembly for moving the jaw members relative to one another from a first position, wherein the jaw members are disposed in spaced relation relative to one another, to a second position, wherein the jaw members cooperate to grasp tissue therebetween; and
a multi-position handle assembly operatively connected to a proximal end of the shaft, the handle assembly pivotable between a first and at least a second gripping position for actuating the drive assembly to move the jaw members.

22. The bipolar forceps according to claim 21, further comprising a source of electrosurgical energy electrically connected to the jaw members such that the jaw members are capable of conducting energy through tissue held therebetween to effect a tissue seal.

23. The bipolar forceps according to claim 22, further comprising a rotating assembly for rotating the jaw members about a longitudinal axis defined by the shaft.
